(19) United States
(12) Patent Application Publication
(10) Pub. No.: US 2007/0203530 A1
(43) Pub. Date: Aug. 30, 2007

(54) Filtered multipolar feedthrough assembly

(76) Inventors: Roger L. Hubing, Hastings, MN (US); Gregory J. Haubrich, Champlin, MN (US); Michael E. Nowak, Andover, MN (US)

Correspondence Address: MEDTRONIC, INC., 710 MEDTRONIC PARKWAY NE, MINNEAPOLIS, MN, 55432-9924, US

(21) Appl. No.: 11/364,263

(22) Filed: Feb. 28, 2006

(51) Int. Cl. A61N 1/375

(52) U.S. Cl. 607/37

(57) ABSTRACT

A filtered feedthrough assembly for use with an implantable medical device includes a ferrule, a plurality of feedthrough conductors extending through the ferrule, and a hermetic seal between the ferrule and the plurality of feedthrough conductors. A filter is electrically connected to a first of the feedthrough conductors to provide a controlled impedance at a reference point located at or near an interior end of the first feedthrough conductor.

### BACKGROUND OF THE INVENTION

The present invention relates to implantable medical devices (IMDs). More particularly, the present invention relates to filtered multipolar feedthrough assemblies for use with IMDs.

Electrical feedthroughs provide a conductive path extending between the interior of a hermetically sealed container and a point outside the container. Some IMDs utilize multipolar electrical feedthroughs that include a plurality of conductive paths that are each connected to internal circuitry for providing therapy, data collection/processing and other functions. Moreover, some IMDs include circuitry for wireless communication, which enables reception and transmission of data in conjunction with an external transceiver. With radio frequency (RF) wireless communication circuitry, an antenna assembly (or RF-passthrough assembly) that extends outside the container (also called an enclosure or case) of the IMD is typically required because the container would otherwise unduly inhibit the transmission of RF communication signals in the Medical Implant Communications Service (MICS) band (402-405 MHz). However, the use of an antenna presents numerous difficulties with respect to the introduction of electromagnetic interference (EMI) into the container of the IMD.

Feedthroughs can be filtered to minimize undesired EMI from entering the container of an IMD and being conducted to sensitive circuitry inside. However, a functioning antenna must receive electromagnetic signals from the environment. As a result, an antenna cannot utilize the same filtering as other feedthroughs for IMDs, and must generally exhibit lower capacitance. Crosstalk (i.e., undesired capacitive, inductive, or conductive coupling from one conductive path to another) between an antenna feedthrough path and other feedthrough paths becomes a significant concern. Even when feedthrough paths are filtered to attenuate EMI as it enters a container, known filters do not inhibit crosstalk at locations along the feedthrough conductor paths that are spaced away from the filter.

In the past, antennae have been incorporated into separate feedthrough assemblies that are physically spaced from other feedthrough assemblies, such as therapy feedthrough assemblies. This increases electromagnetic isolation, and inhibits crosstalk. However, such physical separation takes up valuable space and the need for separate feedthroughs complicates design and assembly of the IMD. An alternative approach has been to provide a multipolar feedthrough, which has one or more of its conductive paths designated as antennae, with shielding provided between the antennae and any other feedthrough paths. However, the shielding required by that approach is bulky and takes up excessive space. Moreover, crosstalk can still occur at unshielded locations along conductor paths, such at near the ends of shielding structures.

### BRIEF SUMMARY OF THE INVENTION

A multi-pole filtering system for multipolar feedthrough assemblies according to the present invention provides a controlled impedance at or near an interior end of a feedthrough conductor of the multipolar feedthrough. A first filter network attenuates electromagnetic interference (EMI) at a first node that is adjacent to the location where a feedthrough conductor enters a case of an implantable medical device, and a second filter network attenuates crosstalk at a second node that is spaced from the first node. The multi-pole filtering system utilizes the parasitic inductance of the feedthrough conductors between the first and second nodes to help attenuate EMI.

### BRIEF DESCRIPTION OF THE DRAWINGS

FIG. 1 is an exploded perspective view of a filtered multipolar feedthrough assembly that includes an electronic module assembly.

FIG. 2 is a cross sectional view of the filtered multipolar feedthrough assembly of FIG. 1.

FIG. 3 is a schematic circuit diagram of an implantable medical device that incorporates the filtered multipolar feedthrough assembly of FIGS. 1 and 2.

FIG. 4 is a schematic cross-sectional view of a portion of an implantable medical device having an alternative filtered multipolar feedthrough assembly.

### DETAILED DESCRIPTION

In general, the present invention provides a filtering system for use with multipolar feedthrough assemblies for implantable medical devices (IMDs). More particularly, the present invention provides a multi-pole or multi-network filtering system for reducing the presence of undesired electromagnetic interference (EMI) at circuitry within the enclosure (also called the case or container) of an IMD. The inventive filtering system provides a controlled impedance at a node located at or near an interior end of a feedthrough conductor of the multipolar feedthrough. Generally, this is accomplished by attenuating EMI at a first node that is adjacent to the location where the feedthrough conductor enters the case of the IMD, and also by attenuating crosstalk (i.e., undesired capacitive, inductive, or conductive coupling from one feedthrough conductor to another) at a second node that is spaced from the first node. In this way, undesired coupling between adjacent conductive paths that occurs beyond the first node (i.e., at a location closer to internal circuitry of the IMD) can be attenuated at the second node. The multi-pole filtering system utilizes the parasitic inductance of the feedthrough conductors between the first and second nodes to help attenuate EMI. This system is particularly useful with multipolar feedthrough assemblies having a low capacitance feedthrough conductor, such as a radio frequency (RF) antenna feedthrough conductor (or RF-passthrough), which otherwise may introduce undesired EMI to internal circuitry due to crosstalk between the low capacitance feedthrough conductor and other feedthrough conductors.

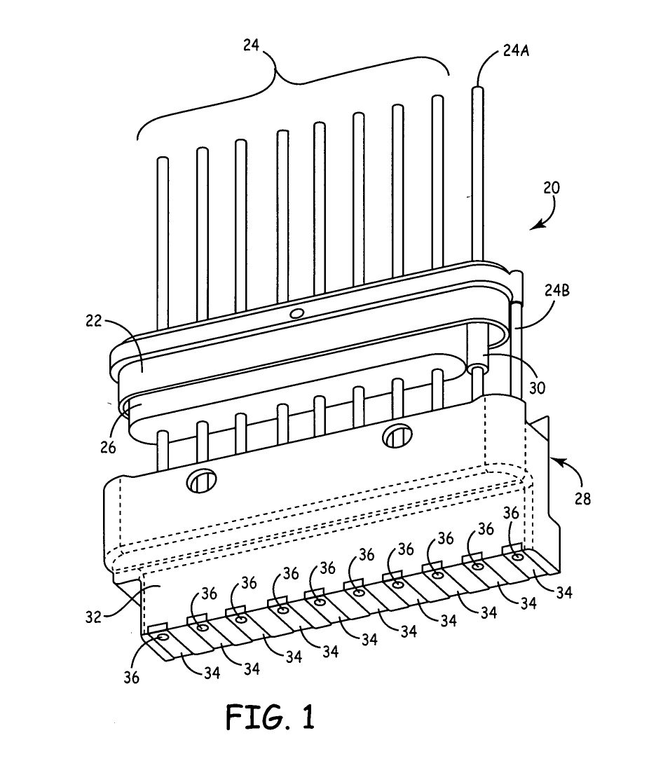

FIG. 1 is an exploded perspective view of a filtered multipolar feedthrough assembly 20 that includes a ferrule 22 (e.g., a conventional titanium ferrule), a plurality of feedthrough conductors 24, an antenna feedthrough conductor 24A, a conductor 24B grounded to the ferrule 22, a monolithic discoidal capacitor assembly 26, and an electronic module assembly (EMA) 28 (also called a molded interconnect device). The feedthrough conductors 24 and the antenna feedthrough conductor 24A are pins that extend through the ferrule 22 and a conventional hermetic seal (not shown) is formed therebetween. The feedthrough conductors 24 can provide conductive paths used for therapy functions. The antenna feedthrough conductor 24A is configured to operate in conjunction with a medical implant communications service (MICS) band transceiver at 402-405 MHz and a conventional external antenna molded within a connector module (not shown). Conventional EMI shielding 30 is optionally provided between a portion of the antenna feedthrough conductor 24A and an adjacent feedthrough conductor 24.

The monolithic discoidal capacitor assembly 26 is positioned around the feedthrough conductors 24, such that the feedthrough conductors 24 extend through openings in the capacitor assembly 26. The capacitor assembly 26 includes discrete discoidal capacitors that are electrically connected to each of the feedthrough conductors 24 at first nodal locations, and are further electrically connected to ground (e.g., being electrically connected to the ferrule 22). The monolithic discoidal capacitor assembly 26 is inserted at least partially within an interior side cavity of the ferrule 22.

The EMA 28 includes a non-conductive body 32, conductive bond pads 34, and openings 36 defined through the body 32 and the bond pads 34. The EMA 28 is positioned over the feedthrough conductors 24, the antenna feedthrough conductor 24A and the grounded conductor 24B at the interior side of the ferrule 22. The feedthrough conductors 24, the antenna feedthrough conductor 24A and the grounded conductor 24B extend through the openings 36 and are electrically connected to the bond pads 34 using welded joints, conductive adhesive, solder, or other suitable electrical connections. The bond pads 34 provide bonding surfaces for electrically connecting wires, ribbons, an other structures to electrically link the feedthrough assembly 20 to circuitry located at the interior side of the assembly 20 (e.g., internal circuitry of an IMD). The bond pads 34 can be made of titanium, nickel/gold or other conductive materials.

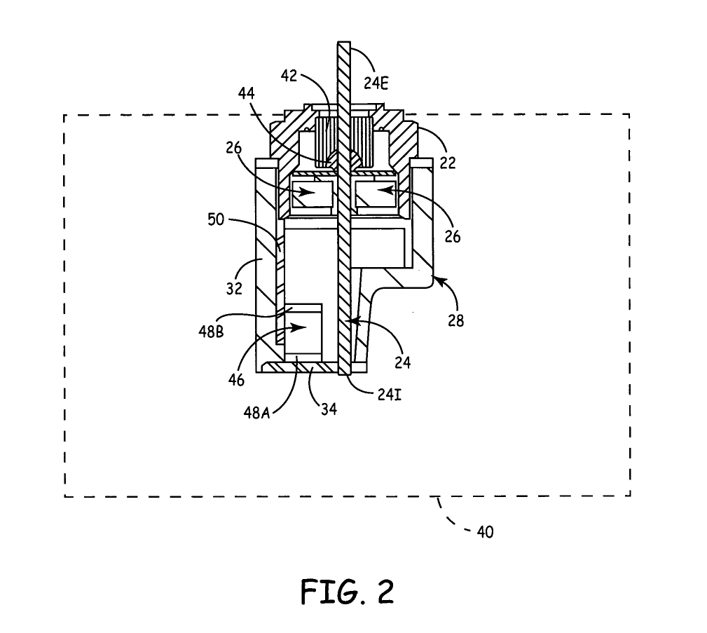

FIG. 2 is a cross sectional view of the filtered multipolar feedthrough assembly 20, with a schematic representation of an IMD case 40 shown in phantom. As illustrated in FIG. 2, the body 32 of the EMA 28 is secured to the ferrule 22 with an epoxy bond. A non-conductive hermetic seal 42 is formed between the feedthrough conductor 24 and the ferrule 22. The seal 42 can comprise a ceramic insulator and gold insulator braze, or another type of known hermetic seal used with IMDs. A conductive gold braze 44 is provided around the feedthrough conductor 24 at an interior side of the hermetic seal 42.

The feedthrough conductor 24 has an interior end 241 and an exterior end 24E. The exterior end 24E can be electrically connected to a connector module (not shown) to facilitate attachment of conventional implantable leads, and the interior end 24I can be electrically connected to circuitry (not shown) inside the case 40. The feedthrough assembly 20 enables operable electrical connections between the implantable leads and the internal circuitry. In one embodiment, the feedthrough conductor 24 is a straight niobium pin having a circular cross-sectional shape, which has an inductance of about 0.9 nanohenrys per millimeter (nH/mm).

A discoidal capacitor 26 is electrically connected to the feedthrough conductor 24 at a location adjacent to the hermetic seal 42 and gold braze 44. Taken in isolation, apart from the rest of the filtering system of the present invention, the discoidal capacitor 26 resembles known single-pole EMI filters for IMDs (it should be noted that the terms “pole”, “element”, and “section” are used synonymously in this context). Generally, it is desirable to electrically connect the discoidal capacitor 26 to the feedthrough conductor 24 at a (first) node that is as close as possible to the point where the feedthrough conductor 24 enters the case 40 through the ferrule 22, in order to provide EMI attenuation before undesired EMI travels any significant distance inside the case 40 along the path formed by the feedthrough conductor 24.

A second capacitor 46 is electrically connected to the feedthrough conductor 24 near its interior end 24I. The second capacitor 46 is a chip capacitor having a first terminal 48A and a second terminal 48B. The first terminal 48A is connected to the bond pad 34 at a location opposite a bonding surface of the bond pad 34. The second terminal 48B is connected to a conductor 50 that is grounded to the ferrule 22. The conductor 50 has a generally planar shape, and can be a conductive foil layer or embedded metal plate located along an interior wall of the body 32 of the EMA 28. The second capacitor 46 is thus electrically connected to the feedthrough conductor 24 at a second node that is spaced from the first node where the discoidal capacitor 26 is electrically connected to the feedthrough conductor 24.

Although FIGS. 1 and 2 illustrate the first capacitor 26 as a discoidal capacitor and the second capacitor 46 as a chip capacitor, it should be recognized that the type and configuration of each capacitor can vary as desired. For instance, both capacitors (26 and 46) can be chip capacitors, or both can be discoidal capacitors. Moreover, the particular values of the capacitors will vary depending on the particular filtering desired for a particular application. In addition, the particular arrangement of electrical connections to the capacitors can vary from those illustrated in FIG. 2.

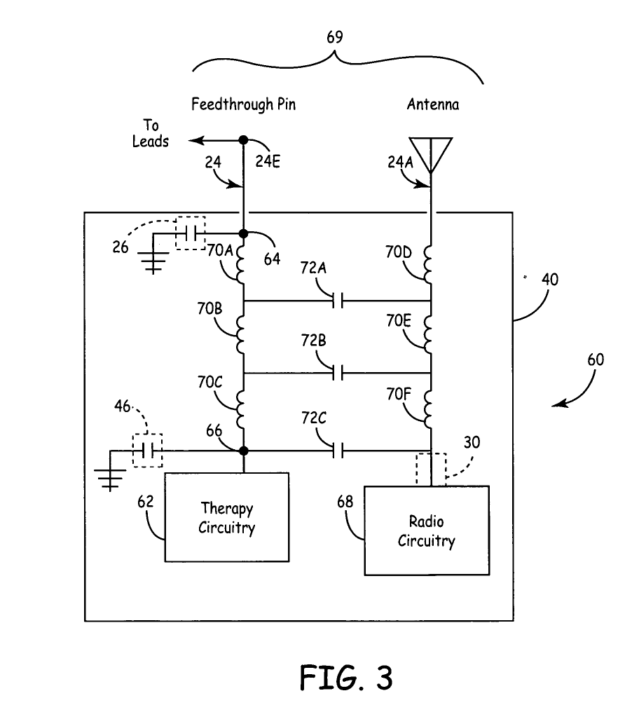

FIG. 3 is a schematic circuit diagram of an implantable medical device 60 that incorporates the filtered multipolar feedthrough assembly shown and described with respect to FIGS. 1 and 2. Only a portion of the filtered multipolar feedthrough assembly is represented in FIG. 3 for simplicity. As shown in FIG. 3, the feedthrough conductor 24 is electrically connected to therapy circuitry 62 located inside the case 40. In further embodiments, the feedthrough conductor can be connected to other types of circuitry, such as data collection/processing circuitry. The first capacitor 26 is electrically connected to the feedthrough conductor 24 at a first node 64, which is located at or near the location where the feedthrough conductor 24 enters the case 40 of the IMD 60, and the second capacitor 46 is electrically connected to the feedthrough conductor 24 at a second node 66, which is located at or near the therapy circuitry 62 and is spaced from the first node 64.

The antenna feedthrough conductor 24A is operably connected to radio circuitry 68 located inside the case 40. The radio circuitry 68 and the therapy circuitry 62 can be incorporated onto separate circuit boards, or combined on a single circuit board inside the case 40. EMI shielding 30, shown in phantom, is optionally provided for portion of the antenna feedthrough conductor 24A. The feedthrough conductor 24 and the antenna feedthrough conductor 24A are collectively referred to as multipolar feedthrough array 69.

Parasitic inductances 70A-70F and are illustrated (i.e., modeled) in FIG. 3. Mutual capacitances 72A-72C are illustrated between the feedthrough conductor 24 and the antenna feedthrough conductor 24A in order to represent crosstalk (i.e., coupling between adjacent conductive paths form by the feedthrough conductor 24 and the antenna feedthrough conductor 24A). Other electrical characteristics of the circuit, such as Ohmic losses, are not shown in FIG. 3 for simplicity. When the lengths of the feedthrough conductor 24 and antenna feedthrough conductor 24A approach the wavelength of signals—desired or undesired—transmitted through the multipolar feedthrough array 69, it can be modeled as a distributed element coupling between the feedthrough conductor 24 and the antenna feedthrough conductor 24A.

It should be noted that the filtering system of the present invention addresses only EMI filtering of the feedthrough conductors 24, but does not directly address EMI filtering of the antenna feedthrough conductor 24A. Separate radio filtering techniques can optionally be used to filter EMI that would otherwise travel along the antenna feedthrough conductor 24A from the antenna to radio circuitry inside an IMD.

In operation, EMI from environmental sources can be conducted by implantable leads connected to external portions of the feedthrough array 69. The capacitor 26 represents a first pole of the filtering system, and acts as a low-pass filter at the first node 64 to attenuate EMI entering the case 40 of the IMD 60. However, crosstalk between the feedthrough conductor 24 and the antenna feedthrough conductor 24A, as well as additional crosstalk between adjacent feedthrough conductors, can introduce undesired EMI to the feedthrough conductor at locations beyond the first node 64, that is, between the first node 64 and the therapy circuitry 62. The capacitor 46 represents a second pole of the filtering system, and acts as a low-pass filter at the second node 66 to attenuate EMI that may have been introduced to the feedthrough conductor 24 due to crosstalk. The filter system shown in FIG. 3 utilizes the parasitic inductance (70A-70C) of the feedthrough conductor 24 to lower the impedance at the second node 66 to enhance filtering. The parasitic inductance (70A-70C, collectively) of the feedthrough conductor 24 represents a third pole of the filtering system. In further embodiments, an inductor, such as a slab inductor, can be electrically connected to the capacitor 46 to enhance filtering independent of the parasitic inductance of the feedthrough conductor 24.

In summary, the present invention provides a feedthrough assembly having multiple filter networks for reducing EMI transmission to internal circuitry. Stated another way, multiple-pole filters are formed where previously only single-pole filters were known. The filtering system of the present invention increases electromagnetic isolation between adjacent conductive paths of a feedthrough array, yet provides a relatively compact design. A first filter network is formed at a first node, utilizing a first capacitor, and a second filter network is formed at a second node, utilizing a second capacitor and the parasitic inductance of the feedthrough conductor. The multi-pole filtering system provides a controlled impedance at the second node.

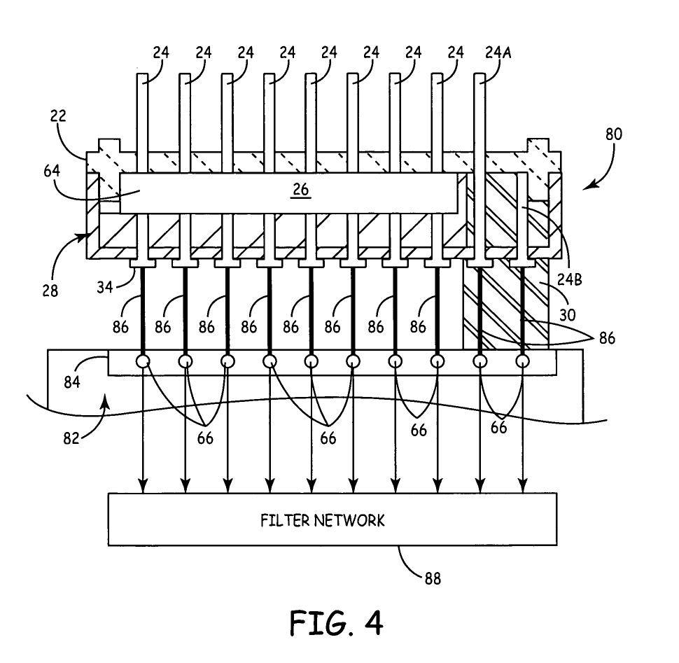

It should be noted that it is generally desirable to locate the second node as close as possible to internal circuitry (e.g., the internal therapy circuitry) so that the filtering system can attenuate as much crosstalk-induced EMI as possible. FIG. 4 is a schematic cross-sectional view of a portion of an implantable medical device utilizing an alternative embodiment of a filtered multipolar feedthrough assembly 80. The assembly 80 includes a feedthrough array 69, an EMA 28, a circuit board 82 having an electrical connection region 84, and wires 86 (e.g., nickel-clad copper ribbons) electrically connected between bond pads 34 of the EMA 28 and the electrical connection region 84 of the circuit board 82. The filtered feedthrough assembly 80 shown in FIG. 4 is generally similar to that shown and described with respect to FIGS. 1-3. However, the second nodes 66 of the assembly 80 shown in FIG. 4 are defined at the electrical connection region 84 of the circuit board 82. In this embodiment, each of the wires 86 can be considered like portions or extensions of the feedthrough conductors 24 and the antenna feedthrough conductor 24A. The second filter network 88 (represented only schematically in FIG. 4) is located as close as possible to the electrical connection regions 84 of the circuit board 82. In that way, the feedthrough assembly 80 promotes attenuation of crosstalk at the interior side of the assembly 80. Such an embodiment is beneficial where the optional shielding 30 is omitted, or where additional electromagnetic isolation is desired—beyond that provided by shielding 30.

Although the present invention has been described with reference to preferred embodiments, workers skilled in the art will recognize that changes may be made in form and detail without departing from the spirit and scope of the invention. For example, the present invention can be utilized with nearly any type of multipolar feedthrough assembly, and is scalable for feedthroughs having any number of conductive paths provided in any arrangement. The feedthrough assembly also need not include a dedicated antenna feedthrough conductor. The present invention can be utilized with any type of multipolar feedthrough assembly having a low-capacitance or unfiltered feedthrough conductor. In addition, the multi-pole filtering system of the present invention can incorporate the use of additional filter poles, such as additional capacitors electrically connected to feedthrough conductors.

1. A filtered feedthrough assembly for use with an implantable medical device, the assembly comprising:
a ferrule;
a plurality of feedthrough conductors extending through the ferrule, wherein each feedthrough conductor has an exterior end and an interior end;
a hermetic seal between the ferrule and the plurality of feedthrough conductors; and
a filter electrically connected to a first of the plurality of feedthrough conductors to provide a controlled impedance at a first reference point located at or near the interior end of the first feedthrough conductor.

2. The assembly of claim 1 and further comprising:
an electronic module assembly block operatively engaged with at least one of the plurality of feedthrough conductors, wherein the filter is supported by the electronic module assembly block.

3. The assembly of claim 1, wherein the filter is a multi-pole filter.

4. The assembly of claim 3, wherein the multi-pole filter comprises:
a first capacitor electrically connected to the first feedthrough conductor at the first reference point; and
a second capacitor electrically connected to the first feedthrough conductor at a second reference point, wherein the second reference point is spaced from the first reference point, and wherein the multi-pole filter utilizes parasitic inductance of the feedthrough conductor between the first and second reference points to provide filtering.

5. The assembly of claim 1, wherein the first feedthrough conductor is electrically connected to a therapy circuit located within the implantable medical device, and wherein another of the plurality of feedthrough conductors is electrically connected to radio circuitry and functions as a radio frequency antenna feedthrough.

6. The assembly of claim 1, wherein the feedthrough conductor comprises a feedthrough pin portion and a connector wire portion.

7. The assembly of claim 1 and further comprising shielding for electromagnetically isolating portions of an adjacent pair of the plurality of feedthrough conductors.

8. A feedthrough assembly for an implantable medical device, the assembly comprising:
a ferrule;
a feedthrough conductor extending through the ferrule;
a hermetic seal between the feedthrough conductor and the ferrule;
a first electromagnetic interference filter network operably connected to the feedthrough conductor at a first node, wherein the first node is located adjacent to the hermetic seal; and
a second electromagnetic interference filter network operably connected to the feedthrough conductor at a second node, wherein the second node is spaced from the first node.

9. The assembly of claim 8, wherein the first electromagnetic interference filter network includes a capacitor that is electrically connected to ground.

10. The assembly of claim 8, wherein the second electromagnetic interference filter network comprises a capacitor electrically connected to ground.

11. The assembly of claim 10, wherein the second electromagnetic interference filter network utilizes parasitic inductance of the feedthrough conductor to provide filtering.

12. The assembly of claim 8, wherein the feedthrough conductor comprises a feedthrough pin portion and a connector wire portion.

13. The assembly of claim 8 and further comprising:
a radio frequency antenna feedthrough that extends through the ferrule and is adjacent to the feedthrough conductor.

14. The assembly of claim 13, wherein the antenna feedthrough conducts signals having a wavelength that approaches the length of the antenna feedthrough.

15. The assembly of claim 13, wherein the second electromagnetic interference filter network is configured to attenuate electromagnetic interference transmitted to the feedthrough conductor due to coupling between the feedthrough conductor and the antenna feedthrough.

16. A filter assembly for use an implantable medical device, the assembly comprising:
a case;
a feedthrough conductor array including a first feedthrough conductor and a second feedthrough conductor that extend between an exterior and an interior of the case, wherein the first feedthrough conductor functions as an antenna feedthrough conductor for conducting radio frequency signals; and
a multi-pole filter electrically connected to the second feedthrough conductor, wherein the multi-pole filter utilizes the parasitic inductance of the second feedthrough conductor to provide filtering.

17. The assembly of claim 16, wherein the second feedthrough conductor comprises a feedthrough pin portion and a connector wire portion.

18. The assembly of claim 16, wherein the multi-pole filter comprises a plurality of capacitors that are each electrically connected to ground.

19. The assembly of claim 16 and further comprising shielding for electromagnetically isolating portions of the first and second feedthrough conductors.

20. The assembly of claim 16, wherein the multi-pole filter comprises a plurality of capacitors positioned at nodes that are spaced apart.
